(19) United States
(12) Patent Application Publication
(10) Pub. No.: US 2024/0389831 A1
(43) Pub. Date: Nov. 28, 2024

(54) MEDICAL IMAGING SYSTEM, MEDICAL IMAGING DEVICE, AND CONTROL METHOD

(71) Applicant: Sony Group Corporation, Tokyo (JP)

(72) Inventor: Yasuaki TAKAHASHI, Tokyo (JP)

(73) Assignee: Sony Group Corporation, Tokyo (JP)

(21) Appl. No.: 18/551,599

(22) PCT Filed: Jan. 25, 2022
(86) PCT No.: PCT/JP2022/002507

§ 371 Date: Feb. 23, 2024

(30) Foreign Application Priority Data
Mar. 31, 2021 (JP) 2021-059287

(51) Int. Cl. A61B 1/00; A61B 1/045; A61B 1/313

(52) U.S. Cl. CPC A61B 1/00045; A61B 1/045; A61B 1/3132

(57) ABSTRACT

The present disclosure relates to a medical imaging system, a medical imaging device, and a control method that enable appropriate outputting of an image according to surgery.

The medical imaging system includes a surgery mode setting unit that sets a surgery mode, and a selection processing unit that performs, on the basis of the surgery mode, selection of switching a display image from among a Near image focused on a near point, a Mid image focused on a middle point, a Far image focused on a far point, and an EDOF image obtained by extending a depth of field by combining those images, the Near image, the Mid image, and the Far image being captured by three pieces of imaging elements having different optical path lengths from one imaging lens. The present technology can be applied to, for example, a medical imaging system capable of capturing an EDOF image.

### TECHNICAL FIELD

The present disclosure relates to a medical imaging system, a medical imaging device, and a control method, and more particularly, to a medical imaging system, medical imaging device, and control method that enable appropriate outputting of an image according to surgery.

### BACKGROUND ART

Conventionally, in a medical field, when work such as surgery is performed while an affected area or the like of an image captured through a lens is observed as a region of interest, regions on a near point side and far point side out of a depth of field are out of focus. For this reason, each time a position of the region of interest shifts to the near point side or the far point side, focus needs to be adjusted so that the region of interest is in focus, and there is a concern that work efficiency decreases in an image with a shallow depth of field. Therefore, there is a demand for a medical imaging system capable of capturing an image with a deep depth of field.

For example, Patent Document 1 discloses a medical observation device capable of acquiring an extended depth of field (EDOF) image obtained by extending a depth of field.

### CITATION LIST

### Patent Document

Patent Document 1: Japanese Patent Application Laid-Open No. 2017-158764

### SUMMARY OF THE INVENTION

### Problems to be Solved by the Invention

By the way, because a required depth of field and focus condition are different depending on a type of surgery, it is required to output an image in which a depth of field and focus are appropriately adjusted according to the surgery.

The present disclosure has been made in view of such a situation, and an object thereof is to enable appropriate outputting of an image according to surgery.

### Solutions to Problems

A medical imaging system and medical imaging device according to one aspect of the present disclosure include a surgery mode setting unit that sets a surgery mode, and a selection processing unit that performs, on the basis of the surgery mode, selection of switching a display image from among two or more types of images captured by at least two pieces of imaging elements having different optical path lengths from one imaging lens, and an EDOF image obtained by extending a depth of field by combining those images.

A control method according to one aspect of the present disclosure includes, by a medical imaging system, setting a surgery mode, and performing, on the basis of the surgery mode, selection of switching a display image from among two or more types of images captured by at least two pieces of imaging elements having different optical path lengths from one imaging lens, and an EDOF image obtained by extending a depth of field by combining those images.

In one aspect of the present disclosure, a surgery mode is set, and, on the basis of the surgery mode, there is performed selection of switching a display image from among two or more types of images captured by at least two pieces of imaging elements having different optical path lengths from one imaging lens, and an EDOF image obtained by extending a depth of field by combining those images.

### BRIEF DESCRIPTION OF DRAWINGS

FIG. 1 is a diagram illustrating a configuration example of an embodiment of a medical imaging system to which the present technology is applied.

FIG. 2 is a diagram describing a configuration of an endoscope and a device unit.

FIG. 3 is a diagram illustrating a configuration example of an imaging module.

FIG. 4 is a diagram for describing a Mid image, a Near image, a Far image, an EDOF image, and a color-coded image.

FIG. 5 is a diagram illustrating a focus position relationship in a cataract surgery mode and a focus position relationship in a vitreous surgery mode.

FIG. 6 is a diagram illustrating a configuration example of an image selection function of a CCU.

FIG. 7 is a flowchart describing image selection processing.

FIG. 8 is a flowchart describing display image output processing.

FIG. 9 is a block diagram illustrating a configuration example of an embodiment of a computer to which the present technology is applied.

### MODE FOR CARRYING OUT THE INVENTION

Hereinafter, a specific embodiment to which the present technology is applied will be described in detail with reference to the drawings.

### <Configuration Example of Medical Imaging System>

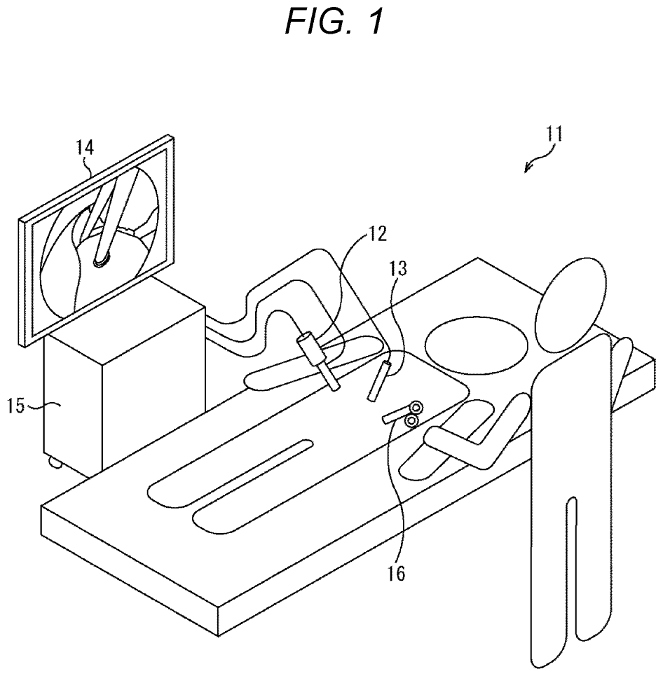

FIG. 1 is a diagram illustrating a configuration example of an embodiment in which a medical imaging system to which the present technology is applied to an endoscopic surgery.

A medical imaging system 11 illustrated in FIG. 1 includes an endoscope 12, an energy treatment tool 13, a display device 14, and a device unit 15.

For example, in a surgery utilizing the medical imaging system 11, the endoscope 12 and the energy treatment tool 13 are inserted into a body of a patient, and forceps 16 are inserted into the body of the patient. Then, in the medical imaging system 11, an image of an affected area such as a tumor imaged by the endoscope 12 is displayed on the display device 14 in real time, and a physician can treat the affected area by using the energy treatment tool 13 and the forceps 16 while viewing the image.

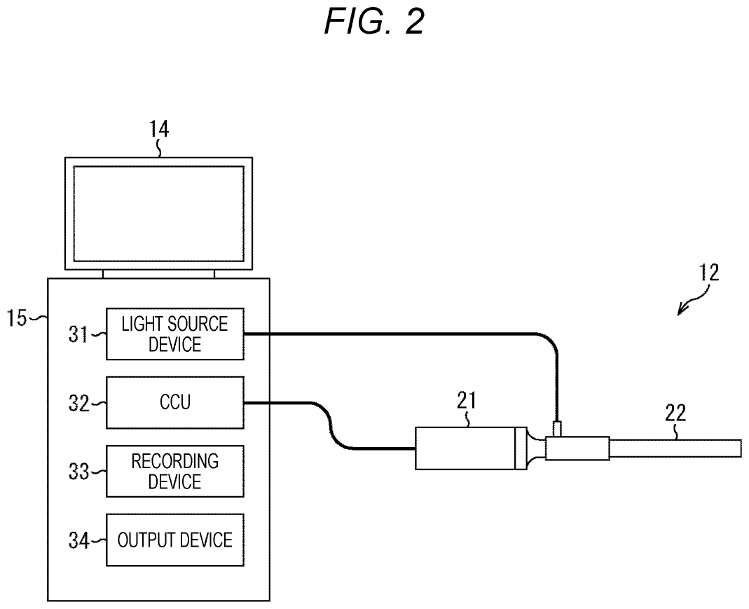

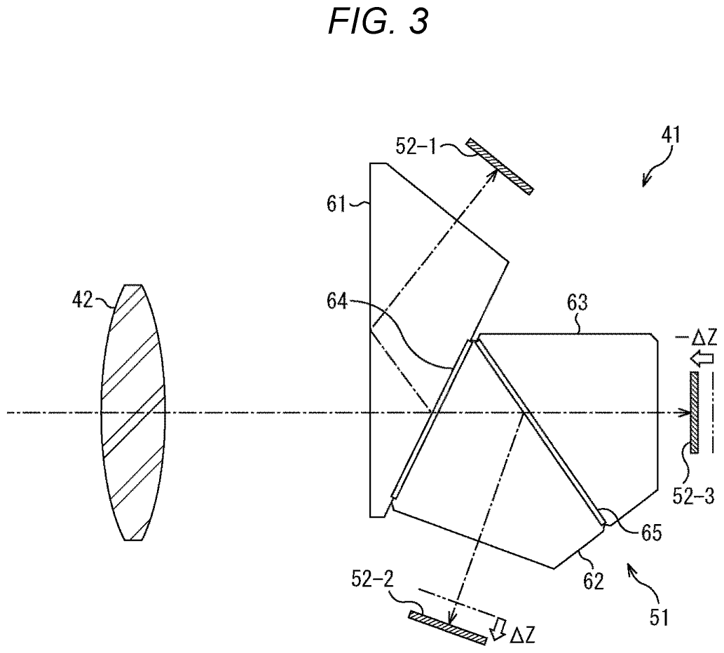

For example, as illustrated in FIG. 2, the endoscope 12 is configured such that a tubular lens barrel unit 22 in which an optical system such as an objective lens is incorporated is mounted on a camera head 21 in which an imaging module (refer to FIG. 3) including a plurality of imaging elements and the like is incorporated. For example, the lens barrel unit 22 is a scope formed into a tubular shape by using a rigid or flexible material, and can guide light to a tip end with a light guide extending inside and emit light on an inside of a body cavity of the patient. The camera head 21 is configured such that an optical element such as, for example, a birefringent mask (BM) can be inserted between the camera head 21 and the lens barrel unit 22, and can capture an image of the inside of the body cavity of the patient via the optical system of the lens barrel unit 22.

The energy treatment tool 13 is, for example, a medical instrument used in an endoscopic surgery, in which an affected area is cut by heat generated by a high-frequency current, or sealing a blood vessel.

The display device 14 can display an image captured by the endoscope 12 as is or can display an image subjected to image processing in the device unit 15.

The device unit 15 is configured by incorporating various devices necessary for performing an endoscopic surgery utilizing the medical imaging system 11. For example, as illustrated in FIG. 2, the device unit 15 can include a light source device 31, a camera control unit (CCU) 32, a recording device 33, and an output device 34.

The light source device 31 supplies, via an optical fiber or the like, the endoscope 12 with light emitted to the affected area when the endoscope 12 performs imaging.

The CCU 32 controls imaging by the endoscope 12 and performs image processing on an image captured by the endoscope 12. Furthermore, the CCU 32 includes, for example, a focus control function for appropriately controlling focus according to a surgery mode when capturing an image with the endoscope 12, an image selection function for appropriately outputting, according to the surgery mode, and the like, an image captured by the endoscope 12.

The recording device 33 records an image output from the CCU 32 on a recording medium. The output device 34 prints and outputs the image output from the CCU 32 or outputs the image via a communication network.

### <Configuration Example of Imaging Module>

FIG. 3 is a diagram illustrating a configuration example of the imaging module incorporated in the camera head 21 of the endoscope 12.

As illustrated in FIG. 3, an imaging module 41 includes an optical splitting system 51 and three imaging elements 52-1 to 52-3. Furthermore, an imaging lens 42 is disposed on an optical axis of light incident on the imaging module 41. The imaging lens 42 includes one or a plurality of pieces of lenses, condenses light toward the imaging elements 52-1 to 52-3 such that imaging by light entering the lens barrel unit 22 of the endoscope 12 is performed, and causes the light to enter the optical splitting system 51.

The optical splitting system 51 splits light incident via the imaging lens 42 toward each of the imaging elements 52-1 to 52-3. The optical splitting system 51 includes a first prism 61, a second prism 62, a third prism 63, a first dichroic mirror 64, and a second dichroic mirror 65.

The first prism 61, the second prism 62, and the third prism 63 include prism blocks between the first prism 61 and the second prism 62 and between the second prism 62 and the third prism 63, the prism blocks being joined so as not to generate an air gap. In this manner, by adopting the prism blocks having a so-called gapless structure, it is possible to avoid catching process dust, oozing sealing material, and the like in the optical splitting system 51. Therefore, in the optical splitting system 51, even with a lens system such as, for example, the endoscope 12 having a relatively large F value, it is possible to eliminate reflection of a foreign matter and reduce deterioration in image quality.

The first dichroic mirror 64 is an optical thin film including a dielectric multilayer film formed against an emission surface of the first prism 61, the emission surface being on a side close to the second prism 62, and the first dichroic mirror 64 splits light such that a ratio of, for example, average reflectance:average transmittance=1:2 in light amount.

The second dichroic mirror 65 is an optical thin film including a dielectric multilayer film formed against an emission surface of the second prism 62, the emission surface being on a side close to the third prism 63, and the second dichroic mirror 65 splits light such that a ratio of, for example, average reflectance:average transmittance=1:1 in light amount.

The imaging elements 52-1 to 52-3 are, for example, CMOS image sensors having a Bayer array RGB filter. The imaging element 52-1 is disposed at a position where a distance (optical path length) from a principal point of the imaging lens 42 is an intermediate distance as a reference. The imaging element 52-2 is disposed at a position away from the optical splitting system 51 by a shift amount ΔZ so that the distance from the principal point of the imaging lens 42 is farther than the reference is. The imaging element 52-3 is disposed at a position close to the optical splitting system 51 by the shift amount ΔZ so that the distance from the principal point of the imaging lens 42 is closer than the reference is.

With this arrangement, in a case where a focal length of the imaging lens 42 is adjusted so that the imaging element 52-1 captures an image focused on a region of interest, the imaging element 52-2 captures an image focused on a point closer to a near point than the region of interest is. Similarly, in this case, the imaging element 52-3 captures an image focused on a point closer to a far point than the region of interest is. Therefore, hereinafter, an image captured by the imaging element 52-1 will be referred to as a Mid image, an image captured by the imaging element 52-2 will be referred to as a Near image, and an image captured by the imaging element 52-3 will be referred to as a Far image as appropriate.

Therefore, the imaging module 41 is configured to be able to output the Near image, the Mid image, and the Far image to the CCU 32.

Then, the medical imaging system 11 can switch the Near image, the Mid image, and the Far image and output the switched image to the display device 14, and can switch an EDOF image subjected to image processing in the CCU 32 and a color-coded image and output the switched image to the display device 14.

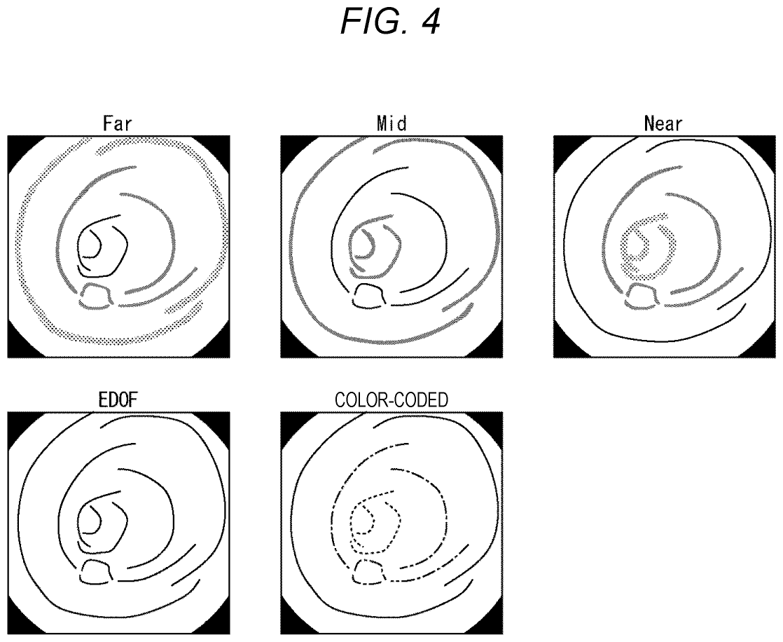

FIG. 4 illustrates images of the Near image, the Mid image, the Far image, the EDOF image, and the color-coded image which are switched and displayed in the medical imaging system 11.

For example, the Near image is captured such that the near point is in focus, in which blur is greater toward a far point side. The Mid image is captured such that a middle point is in focus, in which blur is appearing on the near point side and the far point side. The Far image is captured such that the far point side is in focus, in which blur is greater toward a near point side.

The EDOF image is an image subjected to image processing in which contrast is obtained for each pixel of the Near image, Mid image, and the Far image, and pixels having highest contrast are selected and combined, by which a depth of field is extended so that a range from a near point to a far point is in focus.

The color-coded image is an image obtained by obtaining contrast for each pixel of the Near image, Mid image, and Far image, and color-coding with colors corresponding to the image from which highest contrast is obtained, and the color-coded image is used to select a region. For example, the color-coded image is color-coded such that pixels having highest contrast in the Near image are red (solid lines in FIG. 4), pixels having highest contrast in the Mid image are green (alternate long and short dash lines in FIG. 4), and pixels having highest contrast in the Far image are blue (broken lines in FIG. 4).

Then, in the medical imaging system 11, when a user inputs a surgery mode utilizing, for example, a user interface displayed on the display device 14, the input surgery mode is set in the CCU 32. Then, the CCU 32 can appropriately select and output the Near image, the Mid image, and the Far image, the EDOF image, and the color-coded image according to the set surgery mode.

For example, the user can select which image is to be displayed, by viewing the Near image, the Mid image, the Far image, the EDOF image, and the color-coded image, which are disposed side by side as illustrated in FIG. 4, as options of a display image to be displayed on the display device 14. Note that the display image may be decided by default depending on a surgery mode.

Here, there will be described an example in which a cataract surgery mode, a vitreous surgery mode, and a laparoscope mode, for example, are adopted as surgery modes selectable in the medical imaging system 11. In addition, a corneal surgery mode, the vitreous surgery mode, and a fundus surgery mode may be adopted.

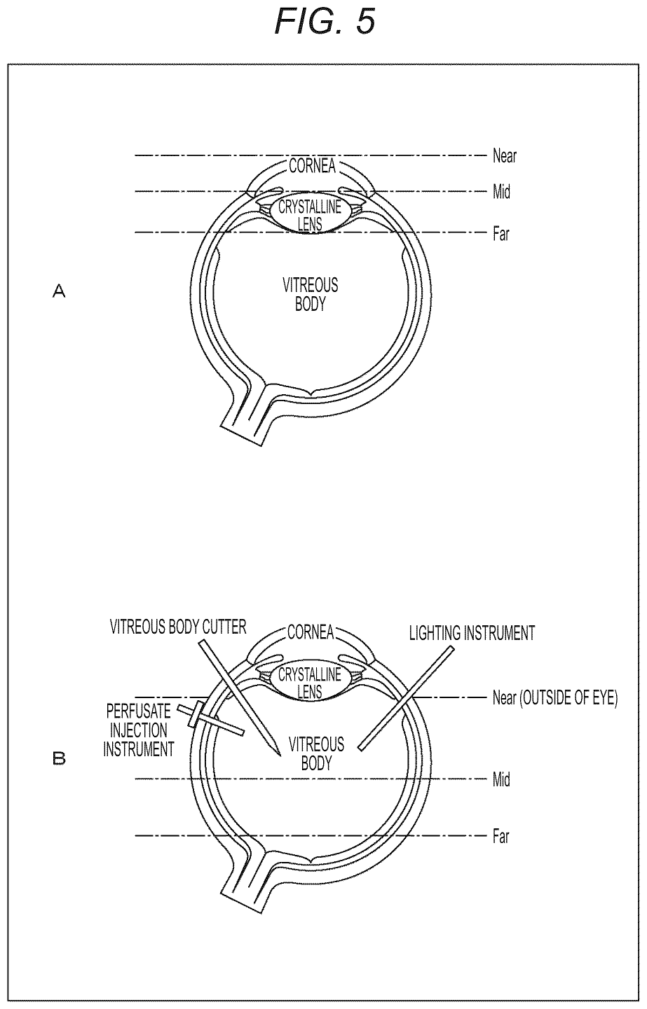

A focus position relationship in the cataract surgery mode and a focus position relationship in the vitreous surgery mode will be described with reference to FIG. 5.

A of FIG. 5 illustrates an example of focus positions of a Near image, a Mid image, and a Far image in the cataract surgery mode.

As illustrated, in the cataract surgery mode, the Near image is captured such that a front surface of a cornea is at the focus position, the Mid image is captured such that a front surface of a crystalline lens is at the focus position, and the Far image is captured such that a back surface of the crystalline lens is at the focus position.

B of FIG. 5 illustrates an example of focus positions of the Mid image, the Near image, and the Far image in the vitreous surgery mode.

As illustrated, in the vitreous surgery mode, the Near image is captured such that an instrument insertion portion outside an eye is at the focus position, the Mid image is captured such that a center of a vitreous body is at the focus position, and the Far image is captured such that a bottom surface of the eye is at the focus position. Here, when the vitreous body is operated on, only the vitreous body is operated without damaging the crystalline lens, and thus, an inside of the vitreous body is viewed with the Mid image and the Far image, and the instrument insertion portion outside the eye is viewed with the Near image.

By the way, in a case where the medical imaging system 11 is used for an ophthalmic surgery, it is preferable to set a difference in optical path length to be small between the near point and the middle point, and to set the difference in optical path length to be large between the middle point and the far point. That is, a difference in optical path length between the imaging element 52-1 and the imaging element 52-2 (ΔZ) is set to be smaller than a difference in the optical path length between the imaging element 52-1 and the imaging element 52-3 (−ΔZ).

With this arrangement, it is possible to easily perform surgery while switching between the Near image and the Mid image near the cornea in an ophthalmic surgery. At this time, it is preferable that the Near image is set such that the vicinity of the surface of the cornea (instrument insertion portion) is in focus, and the Mid image is set a bottom surface of the cornea (an inner side of an eyeball of the cornea) is in focus. Specifically, the difference in optical path length between the imaging element 52-1 and the imaging element 52-2 is preferably set to within 5 mm, because a thickness of a crystalline lens is 4 mm to 5 mm. The difference in optical path length between the imaging element 52-1 and the imaging element 52-3 is preferably set to within 25 mm, because a size of an eyeball from a cornea to a fundus is 22 mm to 23 mm. Note that the optical path length is shorter in order of the imaging element 52-3, the imaging element 52-1, and the imaging element 52-2.

### <Image Selection Function of CCU>

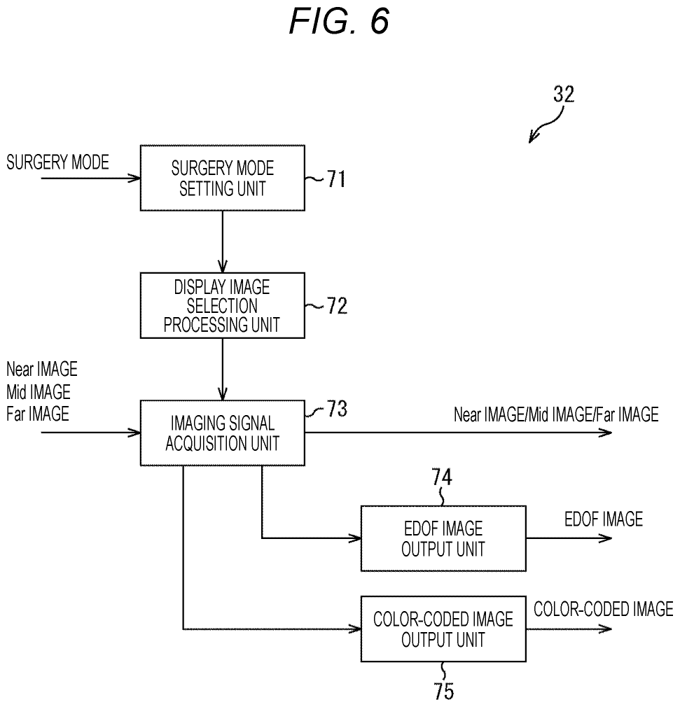

FIG. 6 is a block diagram illustrating an image selection function of the CCU 32.

As illustrated in FIG. 6, the CCU 32 includes a surgery mode setting unit 71, a display image selection processing unit 72, an imaging signal acquisition unit 73, an EDOF image output unit 74, and a color-coded image output unit 75.

For example, when a surgery mode is input by the user by utilizing an input unit (not illustrated), the surgery mode setting unit 71 sets the surgery mode to the display image selection processing unit 72.

The display image selection processing unit 72 presents, on the basis of the surgery mode set by the surgery mode setting unit 71, display image options to be displayed on the display device 14 to the user and when the user selects a desired image to be displayed on the display device 14, the selected image is decided as a display image to be displayed on the display device 14. Furthermore, on the basis of the surgery mode, the display image selection processing unit 72 sets, among the Near image, the Mid image, and the Far image, an EDOF use image to be used by the EDOF image output unit 74 to generate an EDOF image. Furthermore, on the basis of the surgery mode, the display image selection processing unit 72 sets, among the Near image, the Mid image, and the Far image, a base image to be a basis when the color-coded image output unit 75 generates a color-coded image.

The imaging signal acquisition unit 73 acquires imaging signals of the Near image, Mid image, and Far image output from the imaging module 41. Then, the imaging signal acquisition unit 73 outputs the Near image in a case where the Near image is decided as the display image, outputs the Mid image in a case where the Mid image is decided as the display image, and outputs the Far image in a case where the Far image is decided as the display image. Furthermore, in a case where the EDOF use image is decided as the display image, the imaging signal acquisition unit 73 supplies the EDOF image output unit 74 with the EDOF use image set on the basis of the surgery mode. Furthermore, in a case where the color-coded image has been decided as the display image, the imaging signal acquisition unit 73 supplies the Near image, the Mid image, and the Far image to the color-coded image output unit 75, and instructs the color-coded image output unit 75 on the image set as the base image.

The EDOF image output unit 74 generates and outputs an EDOF image by using the EDOF use image supplied from the imaging signal acquisition unit 73. For example, in a case where the Near image, the Mid image, and the Far image are set as EDOF use images, the EDOF image output unit 74 obtains contrast for each pixel of the Near image, Mid image, and Far image. Then, by selecting and combining pixels having highest contrast, the EDOF image output unit 74 generates an EDOF image in which depth of field is extended so that a range from the near point to the far point is in focus.

By using the Near image, Mid image, and Far image supplied from the imaging signal acquisition unit 73, the color-coded image output unit 75 generates and outputs a color-coded image on the basis of the base image set by the display image selection processing unit 72. For example, in a case where the Mid image is instructed as the base image, the color-coded image output unit 75 generates a color-coded image by converting the base image to black and white, obtaining contrast for each pixel of the Near image, Mid image, and Far image, and superimposing colors corresponding to the image from which highest contrast is obtained.

In the CCU 32 configured as described above, when the user inputs a surgery mode, the surgery mode is set in the display image selection processing unit 72 by the surgery mode setting unit 71. Then, the user can set “which region to focus on”, “whether to display EDOF image”, and the like on a screen.

For example, in a case where the cataract surgery mode is set, the display image selection processing unit 72 sets the color-coded image, the EDOF image, the Near image, the Mid image, and the Far image as the display image options. The color-coded image is an option for the user to select, from a plurality of displayed images, which image is better, that is, where in the image needs to be in focus.

Note that, in a case where the cataract surgery mode is set, the display image options may be displayed as “selection”, “surgical tool”, “cornea”, “anterior capsule”, and “posterior capsule”, and the names of the options may be changed according to the surgery mode. However, even if the names of the options are changed according to the surgery mode (for example, if an image name “cornea” in the cataract surgery mode is changed to “vitreous body” in the vitreous surgery mode), the optical path length remains as is.

In the medical imaging system 11, it is preferable that the user can switch the selection by utilizing a switch (for example, a foot pedal or the like), voice input, or the like. With this arrangement, the focus can be changed more quickly than when the focus is adjusted during a surgery, and efficiency of the surgery can be improved.

Furthermore, in a case where the cataract surgery mode is set, the color-coded image generated by the color-coded image output unit 75 is used to select a region, and the Mid image is set as a base image on which the color-coded image is generated. That is, the image displayed when selecting the region is the Mid image, the user designates which region of the Mid image to be brought into focus, and the color-coded image output unit 75 generates an image in which the designated region is estimated to be most in focus (outlines are sharpest).

Furthermore, in a case where the cataract surgery mode is set, the Near image, the Mid image, and the Far image are set as the EDOF use images used by the EDOF image output unit 74 to generate an EDOF image. This is because a cataract surgery is often performed while viewing an instrument insertion portion, a vicinity of a cornea, and a vicinity of a vitreous body. That is, the instrument insertion portion is viewed in the Near image, the vicinity of the cornea as a surgery target is viewed in the Mid image, and whether or not the vitreous body is affected is monitored in the Far image. Note that the images used for the EDOF images for each surgery mode may be changeable by the user.

When presenting the display image options to the user, the display image selection processing unit 72 may highlight, with a red frame or the like, an image being displayed on the display device 14 or an image recommended on the basis of a preset. Note that, when the display image options are presented to the user, the display image options can be displayed on a display different from the display device 14, as a user interface for selecting a display image.

For example, the user can decide which image to display by viewing the Near image, the Mid image, the Far image, the EDOF image, and the color-coded image (image for region selection) disposed side by side as illustrated in FIG. 4. Note that the display image may be decided by default depending on a surgery mode. In the color-coded image (image for region selection), each region is color-coded so as to correspond to the Near image, the Mid image, and the Far image, and, with touch input of a portion in corresponding color in the color-coded image for example, the user can select which area in the image the user wishes to view in focus.

Furthermore, the CCU 32 may recognize a region, such as the cornea, with image recognition so that the cornea is in focus in the Mid image or the Near image. Furthermore, when the EDOF image is selected, the screen may have a potion on which EDOF is performed and a portion on which the EDOF is not performed. For example, the Mid image may be output at all times for the center of the screen, and the EDOF may be performed on the Mid image and Far image for periphery of the center. Note that how far the center of the screen is set can be performed by a previous setting or image recognition. In this manner, the user can perform a setting for EDOF in advance.

Note that, also in a case where the vitreous surgery mode is set, expression of options to be presented to the user and images to be combined in EDOF can be differentiated. Furthermore, an image set as the base image may be arbitrarily changed by the user.

Furthermore, the surgery mode is preferably created from a viewpoint of a surgery target such as a cornea, an iris, a conjunctiva, a crystalline lens, a ciliary body, a vitreous body, an optic papilla, and a macular area, or from a viewpoint of a type of surgery (operative method) (for example, a tumor excision, a pterygium excision, an ophthalmic ptosis surgery, a strabismus correction surgery, a corneal transplant, a corneal refractive correction surgery, a cataract surgery, a glaucoma surgery, a retinal detachment surgery, a vitreous surgery, an intraocular lens (IOL) insertion surgery, and the like). Then, the user selects a surgery target or an operative method to decide a surgery mode.

The image selection function of the CCU 32 is configured as described above, and an image can be appropriately switched and output to the display device 14 according to the surgery mode, in a case where a required depth of field condition is different, in a case where it is necessary to switch and view a plurality of imaging signals having different focus positions, or the like.

For example, in a surgery of an anterior eye, a depth of field capable of bringing the anterior eye into focus is sufficient. However, there is also a scene where a port, a fundus, or the like is temporarily viewed, and conventionally, it is necessary to perform focus adjustment that takes time and labor each time. Furthermore, in a surgery of an anterior eye, it is necessary to switch and view imaging signals in focus, because a cornea, a crystalline lens, and the like are transparent, and, conventionally, it is necessary to adjust focus each time in a case where a depth of field is shallow.

On the other hand, in the medical imaging system 11, the Near image, the Mid image, and the Far image having different focus positions can be simultaneously acquired, and the display image to be output to the display device 14 can be switched according to the surgery mode. Furthermore, in the medical imaging system 11, it is possible to generate a more optimal EDOF image by switching the EDOF use image according to the surgery mode.

With this arrangement, for example, in a surgery of an anterior eye, the medical imaging system 11 simultaneously acquires images focused on an anterior eye, a port, and a fundus, and switches a display image to be output to the display device 14, by which the user can quickly view the image without adjusting focus. Therefore, the medical imaging system 11 can achieve a required depth of field extension and switching of imaging signals having different focus positions without focus adjustment, according to a surgery mode.

Furthermore, because light does not reach a back of an endoscope conventionally, visibility may be poor at the back even in a case where a depth of field is deep. On the other hand, by performing EDOF using the three pieces of imaging elements 52-1 to 52-3, the medical imaging system 11 can improve the visibility as compared to, for example, EDOF using a phase mask, and can improve a degree of hue or depth of field extension.

### <Processing Example of Image Selection Processing>

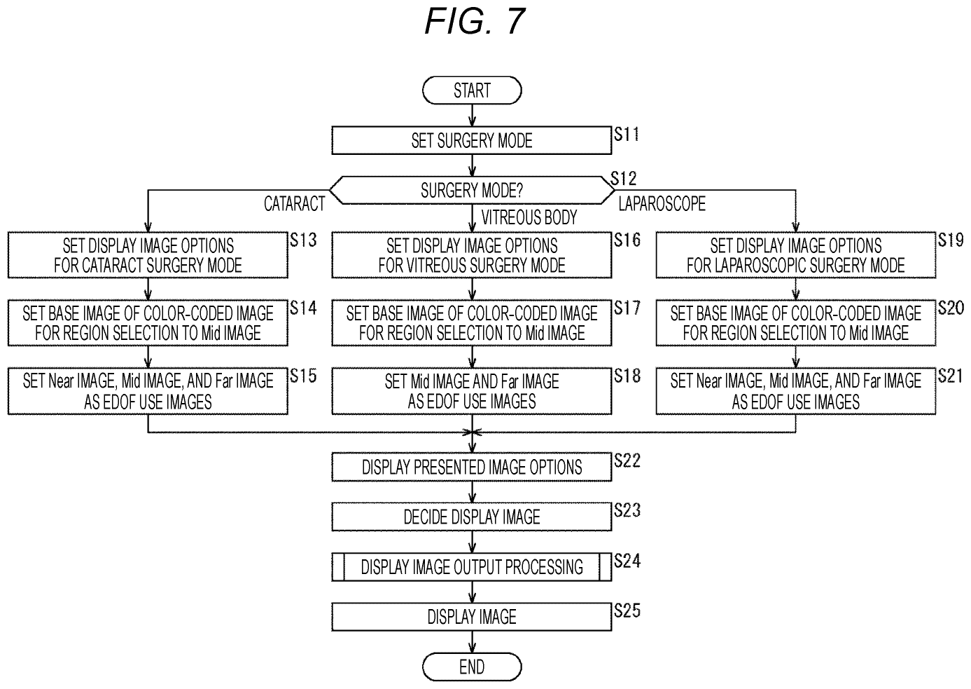

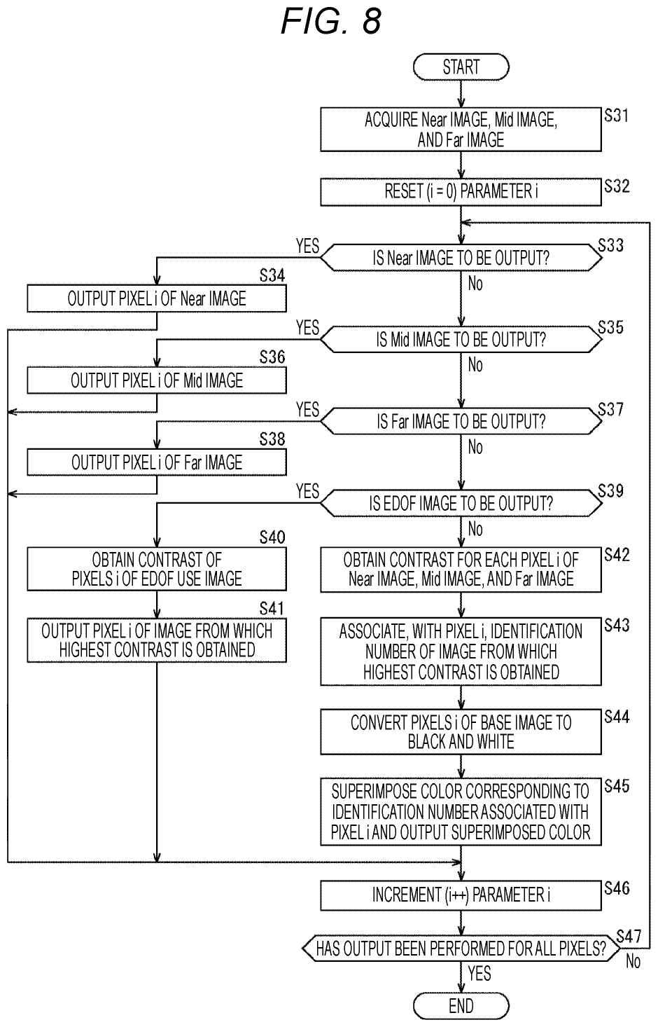

Image selection processing will be described with reference to flowcharts illustrated in FIGS. 7 and 8.

FIG. 7 is a flowchart describing image selection processing executed by the CCU 32. Note that there will be described in FIG. 7 a processing example in which any one of the cataract surgery mode, the vitreous surgery mode, and a laparoscopic surgery mode is set as the surgery mode, but similar processing is performed in a case where various surgery modes as described above are set.

For example, when a surgery mode is input by the user, processing is started, and in Step S11, the surgery mode setting unit 71 sets the surgery mode input by the user to the display image selection processing unit 72.

In Step S12, the display image selection processing unit 72 determines whether the surgery mode set in Step S11 is the cataract surgery mode, the vitreous surgery mode, or the laparoscopic surgery mode.

In a case where the display image selection processing unit 72 determines in Step S12 that the cataract surgery mode has been set, the processing proceeds to Step S13.

In Step S13, the display image selection processing unit 72 sets a color-coded image, an EDOF image, a Near image, a Mid image, and a Far image as display image options for the cataract surgery mode. In Step S14, the display image selection processing unit 72 sets a base image, which serves as a base for when generating a color-coded image, to the Mid image, and in Step S15, sets the Near image, the Mid image, and the Far image as the EDOF use images.

Meanwhile, in a case where the display image selection processing unit 72 determines in Step S12 that the vitreous surgery mode has been set, the processing proceeds to Step S16.

In Step S16, the display image selection processing unit 72 sets the color-coded image, the EDOF image, the Near image, the Mid image, and the Far image as display image options for the vitreous surgery mode. In Step S17, the display image selection processing unit 72 sets a base image, which serves as a base for when generating a color-coded image, to the Mid image, and in Step S18, sets the Mid image and the Far image as the EDOF use images.

Meanwhile, in a case where the display image selection processing unit 72 determines in Step S12 that the laparoscopic surgery mode has been set, the processing proceeds to Step S19.

In Step S19, the display image selection processing unit 72 sets the color-coded image, the EDOF image, and the Mid image as display image options for the laparoscopic surgery mode. In Step S20, the display image selection processing unit 72 sets a base image, which serves as a base for when generating a color-coded image, to the Mid image, and in Step S18, sets the Near image, the Mid image, and the Far image as the EDOF use images.

After the processing in Step S15, Step S18, or Step S21, the processing proceeds to Step S22, and the display image selection processing unit 72 displays the display image options set in Step S13, Step S16, or Step S19 and presents the display image options to the user.

In Step S23, when the user selects, from among the display image options presented in Step S22, a desired image to be displayed on the display device 14, the display image selection processing unit 72 decides the selected image as a display image to be displayed on the display device 14.

In Step S24, display image output processing (refer to FIG. 8 described later) for outputting the display image decided in Step S23 is executed.

In Step S25, after the image output in the display image output processing in Step S24 is displayed on the display device 14, the processing ends.

FIG. 8 is a flowchart describing the display image output processing executed in Step S24 in FIG. 7.

In Step S31, the imaging signal acquisition unit 73 acquires imaging signals of the Near image, Mid image, and Far image output from the imaging module 41.

In Step S32, the imaging signal acquisition unit 73 resets (i=0) a parameter i that specifies a pixel to be processed.

In Step S33, the imaging signal acquisition unit 73 determines whether or not it is decided in Step S23 in FIG. 7 that the Near image is to be output as a display image, and in a case where it is determined that it is decided that the Near image is to be output, the processing proceeds to Step S34. In Step S34, the imaging signal acquisition unit 73 outputs a pixel i of the Near image.

Meanwhile, in a case where the imaging signal acquisition unit 73 determines in Step S33 that it is not decided to output the Near image as a display image, that is, in a case where an image other than the Near image is decided as a display image, the processing proceeds to Step S35.

In Step S35, the imaging signal acquisition unit 73 determines whether or not it is decided in Step S23 in FIG. 7 that the Mid image is to be output as a display image, and in a case where it is determined that it is decided that the Mid image is to be output, the processing proceeds to Step S36. In Step S36, the imaging signal acquisition unit 73 outputs a pixel i of the Mid image.

Meanwhile, in a case where the imaging signal acquisition unit 73 determines in Step S35 that it is not decided to output the Mid image as a display image, that is, in a case where an image other than the Mid image is decided as a display image, the processing proceeds to Step S37.

In Step S37, the imaging signal acquisition unit 73 determines whether or not it is decided in Step S23 in FIG. 7 that the Far image is to be output as a display image, and in a case where it is determined that it is decided that the Far image is to be output, the processing proceeds to Step S38. In Step S38, the imaging signal acquisition unit 73 outputs a pixel i of the Far image.

Meanwhile, in a case where the imaging signal acquisition unit 73 determines in Step S37 that it is not decided to output the Far image as a display image, that is, in a case where an image other than the Far image is decided as a display image, the processing proceeds to Step S39.

In Step S39, the imaging signal acquisition unit 73 determines whether or not it is decided in Step S23 in FIG. 7 that the EDOF image is to be output as a display image, and in a case where it is determined that it is decided that the EDOF image is to be output, the processing proceeds to Step S40.

In Step S40, the imaging signal acquisition unit 73 supplies the EDOF image output unit 74 with the EDOF use image set in Step S15, Step S18, or Step S21 in FIG. 7. The EDOF image output unit 74 obtains contrast of the pixel i for the EDOF use image supplied from the imaging signal acquisition unit 73.

In Step S41, among contrast of pixels i of the EDOF use image obtained in Step S40, the EDOF image output unit 74 outputs the pixel i of an image from which highest contrast is obtained.

Meanwhile, in a case where the imaging signal acquisition unit 73 determines in Step S39 that it is not decided in Step S23 in FIG. 7 to output the EDOF image as a display image, the processing proceeds to Step S42. In this case, in consideration of the determinations in Steps S33, S35, and S37, it is decided in Step S23 in FIG. 7 to output the color-coded image as a display image.

In Step S42, the color-coded image output unit 75 obtains contrast for each pixel i of the Near image, Mid image, and Far image supplied from the imaging signal acquisition unit 73.

In Step S43, among contrast of pixels i of the Near image, Mid image, and Far image obtained in Step S42, the color-coded image output unit 75 associates, with the pixel i, an identification number that identifies an image from which highest contrast is obtained.

In Step S44, the color-coded image output unit 75 converts pixels i of the base image to black and white.

In Step S45, the color-coded image output unit 75 superimposes color corresponding to the identification number associated with the pixel i in Step S43, on the pixels i converted to black and white in Step S44, and outputs the superimposed color.

In Step S46, a selection map generation unit 77 increments (i++) the parameter i that specifies the pixels to be processed.

In Step S47, the imaging signal acquisition unit 73 determines whether or not output has been performed for all the pixels. For example, in a case where the parameter i matches the number of pixels, the imaging signal acquisition unit 73 can determine that the output has been performed for all the pixels.

In a case where the imaging signal acquisition unit 73 determines in Step S47 that output has not been performed for all the pixels, the processing returns to Step S33, and similar processing is repeatedly performed thereafter.

Meanwhile, in a case where the imaging signal acquisition unit 73 determines in Step S47 that the output has been performed for all the pixels, the processing ends.

As described above, in the medical imaging system 11, an image can be appropriately switched and output according to the surgery mode.

Note that, in the present embodiment, a configuration example using three pieces of imaging elements 52-1 to 52-3 has been described. However, for example, the present technology can be applied to a configuration using at least two pieces of imaging elements 52-1 and 52-2. In such a configuration, for example, three types of images, a Near image, a Mid image, and an EDOF image generated from the Near image and Mid image can be selectively output according to the surgery mode.

### <Configuration Example of Computer>

Next, the series of processes (control method) described above can be performed by hardware or software. In a case where a series of processing is performed by software, a program that constitutes the software is installed on a general-purpose computer, or the like.

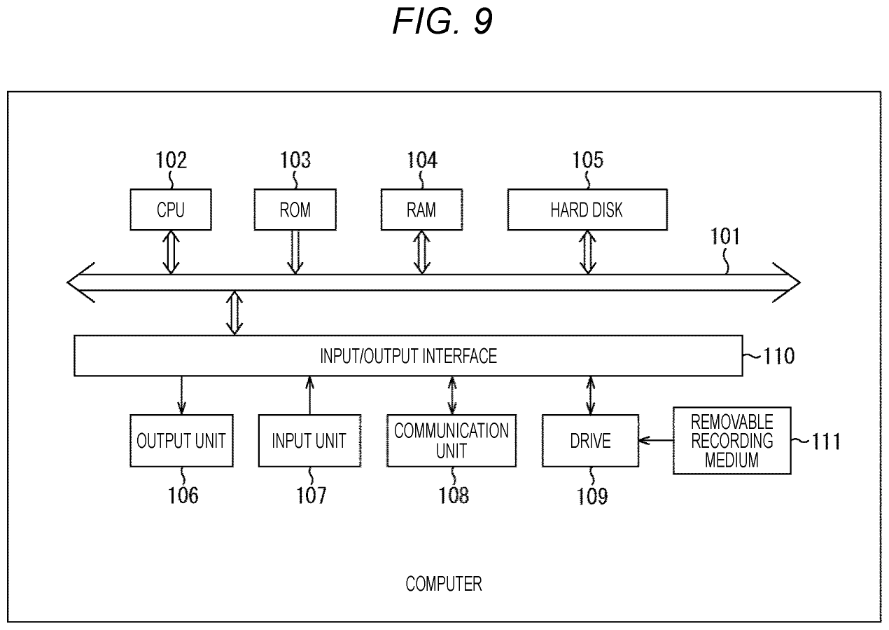

FIG. 9 is a block diagram illustrating a configuration example of an embodiment of a computer on which a program for executing the above-described series of processing is installed.

The program can be previously recorded on a hard disk 105 or ROM 103 as a recording medium incorporated in the computer.

Alternatively, the program can be stored (recorded) in a removable recording medium 111 driven by a drive 109. Such a removable recording medium 111 can be provided as so-called package software. Here, examples of the removable recording medium 111 include, for example, a flexible disk, a compact disc read only memory (CD-ROM), a magneto optical (MO) disk, a digital versatile disc (DVD), a magnetic disk, a semiconductor memory, and the like.

Note that in addition to installing the program on the computer from the removable recording medium 111 as described above, the program can be downloaded to the computer via a communication network or a broadcasting network and installed on the incorporated hard disk 105. That is, for example, the program can be wirelessly transferred from a download site to the computer via an artificial satellite for digital satellite broadcasting, or can be transferred by wire to the computer via a network such as a local area network (LAN) and the Internet.

The computer has a built-in central processing unit (CPU) 102, and an input/output interface 110 is connected to the CPU 102 via a bus 101.

When a command is inputted by a user operating an input unit 107 or the like via the input/output interface 110, in response to this, the CPU 102 executes a program stored in the read only memory (ROM) 103. Alternatively, the CPU 102 loads a program stored in the hard disk 105 into a random access memory (RAM) 104 and executes the program.

With this arrangement, the CPU 102 performs processing according to the above-described flowchart or processing performed according to the above configuration described with the block diagram. Then, as necessary, the CPU 102 outputs a processing result from an output unit 106, transmits the processing result from a communication unit 108, causes the hard disk 105 to record the processing result, or the like, via the input/output interface 110, for example.

Note that the input unit 107 includes a keyboard, a mouse, a microphone, or the like. Furthermore, the output unit 106 includes a liquid crystal display (LCD), a speaker, and the like.

Here, in this specification, the processing to be performed by the computer in accordance with the program is not necessarily performed in chronological order according to the sequences described in the flowcharts. That is, the processing to be performed by the computer in accordance with the program includes processing to be executed in parallel or independently of one another (parallel processing or object-based processing, for example).

Furthermore, the program may be processed by one computer (processor) or processed in a distributed manner by a plurality of computers. Moreover, the program may be transferred to a distant computer and executed.

Moreover, in the present description, a system means a set of a plurality of components (devices, modules (parts), and the like), and it does not matter whether or not all components are in the same housing. Therefore, a plurality of devices housed in separate housings and connected via a network, and one device in which a plurality of modules is housed in one housing are both systems.

Furthermore, for example, it is also possible to divide the configuration described as one device (or processing unit) into a plurality of devices (or processing units). Other way round, it is also possible to put the configurations described above as a plurality of devices (or processing units) together as one device (or processing unit). Furthermore, a configuration other than the above-described configurations may be added to the configuration of each device (or each processing unit). Moreover, when the configuration and operation of the entire system are substantially the same, part of the configuration of a certain device (or processing unit) may be included in the configuration of another device (or another processing unit).

Furthermore, for example, the present technology can be configured as cloud computing in which one function is shared and jointly processed by a plurality of devices via a network.

Furthermore, for example, the above-described program can be executed by an arbitrary device. In this case, it is sufficient that the device has a necessary function (functional block or the like) and is only required to obtain necessary information.

Furthermore, for example, respective steps described in the above-described flowcharts can be executed by one device or can be executed in a shared manner by a plurality of devices. Moreover, in a case where a plurality of processing is included in one step, the plurality of processing included in the one step can be executed by a single device or shared and executed by a plurality of devices. In other words, a plurality of pieces of processing included in one step may be executed as processes of a plurality of steps. Conversely, the processing described as a plurality of steps can be collectively executed as one step.

Note that, the program executed by the computer may be a program in which processing in steps describing the program is executed in time series in an order described in the present specification, or a program in which the processing may be executed in parallel, or individually at a necessary timing such as when a call is made. That is, unless there is a contradiction, the processing of each step may be executed in an order different from the order described above. Moreover, this processing in steps describing program may be executed in parallel with processing of another program, or may be executed in combination with processing of another program.

Note that a plurality of pieces of the present technology which has been described in the present description can each be implemented independently as a single unit as long as no contradiction occurs. Needless to say, a plurality of arbitrary pieces of the present technology can be used in combination. For example, part or all of the present technology described in any of the embodiments can be implemented in combination with part or all of the present technologies described in other embodiments. Furthermore, part or all of any of the above-described present technology can be implemented using together with another technology that is not described above.

### Examples of Configuration Combinations

Note that the present technology can also have the following configuration.

(1)

A medical imaging system including

a surgery mode setting unit that sets a surgery mode, and

a selection processing unit that performs, on the basis of the surgery mode, selection of switching a display image from among two or more types of images captured by at least two pieces of imaging elements having different optical path lengths from one imaging lens, and an extended depth of field (EDOF) image obtained by extending a depth of field by combining those images.

(2)

The medical imaging system according to (1),

in which a Near image focused on a near point, a Mid image focused on a middle point, and a Far image focused on a far point are used, the Near image, the Mid image, and the Far image being captured by three pieces of imaging elements having different optical path lengths from one imaging lens.

(3)

The medical imaging system according to (2),

in which the selection processing unit performs, on the basis of the surgery mode, selection of switching a display image from among the Near image, the Mid image, the Far image, the EDOF image obtained by combining the Near image and the Mid image, the EDOF image obtained by combining the Mid image and the Far image, and the EDOF image obtained by combining the Near image, the Mid image, and the Far image.

(4)

The medical imaging system according to (2) or (3),

in which, regarding optical path lengths of a first imaging element that captures the Mid image, a second imaging element that captures the Near image, and a third imaging element that captures the Far image, the optical path lengths being from an imaging lens, a difference in optical path length between the first imaging element and the second imaging element is smaller than a difference in optical path length between the first imaging element and the third imaging element.

(5)

The medical imaging system according to any one of (2) to (4), the medical imaging system further including an EDOF image generation unit that generates an extended depth of field (EDOF) image obtained by obtaining contrast for each pixel of the Near image, Mid image, and Far image, and selecting and combining pixels having highest contrast.

(6)

The medical imaging system according to any one of (2) to (5), the medical imaging system further including a color-coded image generation unit that generates a color-coded image obtained by setting any one of the Near image, the Mid image, and the Far image as a base image, obtaining contrast for each pixel of the Near image, Mid image, and Far image, and superimposing, on the base image, a color corresponding to an image from which highest contrast is obtained.

(7)

The medical imaging system according to any one of (1) to (6),

in which, as the surgery mode, a cataract surgery mode, a vitreous surgery mode, and a laparoscope mode are set.

(8)

A medical imaging device including

a surgery mode setting unit that sets a surgery mode, and

a selection processing unit that performs, on the basis of the surgery mode, selection of switching a display image from among two or more types of images captured by at least two pieces of imaging elements having different optical path lengths from one imaging lens, and an extended depth of field (EDOF) image obtained by extending a depth of field by combining those images.

(9)

A control method including, by a medical imaging system

setting a surgery mode, and

performing, on the basis of the surgery mode, selection of switching a display image from among two or more types of images captured by at least two pieces of imaging elements having different optical path lengths from one imaging lens, and an extended depth of field (EDOF) image obtained by extending a depth of field by combining those images.

Note that the present embodiment is not limited to the above-described embodiment, and various modifications can be made without departing from the gist of the present disclosure. Furthermore, the effects described in the present specification are merely examples and are not limited, and other effects may be provided.

### REFERENCE SIGNS LIST

11 Medical imaging system
12 Endoscope
13 Energy treatment tool
14 Display device
15 Device unit
16 Forceps
21 Camera head
22 Lens barrel unit
31 Light source device
32 CCU
33 Recording device
34 Output device
41 Imaging module
42 Imaging lens
51 Optical splitting system
52-1 to 52-3 Imaging element
61 First prism
62 Second prism
63 Third prism
64 First dichroic mirror
65 Second dichroic mirror
71 Surgery mode setting unit
72 Region-of-interest setting unit
73 Imaging signal acquisition unit
74 EDOF image output unit
75 Color-coded image output unit

1. A medical imaging system comprising:
a surgery mode setting unit that sets a surgery mode; and
a selection processing unit that performs, on a basis of the surgery mode, selection of switching a display image from among two or more types of images captured by at least two pieces of imaging elements having different optical path lengths from one imaging lens, and an extended depth of field (EDOF) image obtained by extending a depth of field by combining those images.

2. The medical imaging system according to claim 1,
wherein a Near image focused on a near point, a Mid image focused on a middle point, and a Far image focused on a far point are used, the Near image, the Mid image, and the Far image being captured by three pieces of imaging elements having different optical path lengths from one imaging lens.

3. The medical imaging system according to claim 2,
wherein the selection processing unit performs, on a basis of the surgery mode, selection of switching a display image from among the Near image, the Mid image, the Far image, the EDOF image obtained by combining the Near image and the Mid image, the EDOF image obtained by combining the Mid image and the Far image, and the EDOF image obtained by combining the Near image, the Mid image, and the Far image.

4. The medical imaging system according to claim 2,
wherein, regarding optical path lengths of a first imaging element that captures the Mid image, a second imaging element that captures the Near image, and a third imaging element that captures the Far image, the optical path lengths being from an imaging lens, a difference in optical path length between the first imaging element and the second imaging element is smaller than a difference in optical path length between the first imaging element and the third imaging element.

5. The medical imaging system according to claim 2, the medical imaging system further comprising an EDOF image generation unit that generates an extended depth of field (EDOF) image obtained by obtaining contrast for each pixel of the Near image, Mid image, and Far image, and selecting and combining pixels having highest contrast.

6. The medical imaging system according to claim 2, the medical imaging system further comprising a color-coded image generation unit that generates a color-coded image obtained by setting any one of the Near image, the Mid image, and the Far image as a base image, obtaining contrast for each pixel of the Near image, Mid image, and Far image, and superimposing, on the base image, a color corresponding to an image from which highest contrast is obtained.

7. The medical imaging system according to claim 1,
wherein, as the surgery mode, a cataract surgery mode, a vitreous surgery mode, and a laparoscope mode are set.

8. A medical imaging device comprising:
a surgery mode setting unit that sets a surgery mode; and
a selection processing unit that performs, on a basis of the surgery mode, selection of switching a display image from among two or more types of images captured by at least two pieces of imaging elements having different optical path lengths from one imaging lens, and an extended depth of field (EDOF) image obtained by extending a depth of field by combining those images.

9. A control method comprising, by a medical imaging system:
setting a surgery mode; and
performing, on a basis of the surgery mode, selection of switching a display image from among two or more types of images captured by at least two pieces of imaging elements having different optical path lengths from one imaging lens, and an extended depth of field (EDOF) image obtained by extending a depth of field by combining those images.
